(19) United States
(12) Patent Application Publication
(10) Pub. No.: US 2010/0158750 A1
(43) Pub. Date: Jun. 24, 2010

(54) APPARATUS AND METHOD FOR DE-ODORISING A GAS MIXTURE

(76) Inventors: Beng Lai Koh, Singapore (SG); Thim Sui Chung, Singapore (SG)

Correspondence Address: SNELL & WILMER L.L.P. (Main), 400 EAST VAN BUREN, ONE ARIZONA CENTER, PHOENIX, AZ, 85004-2202, US

(21) Appl. No.: 12/668,723

(22) PCT Filed: Jul. 12, 2007
(86) PCT No.: PCT/SG07/00210

§ 371 Date: Jan. 12, 2010

(51) Int. Cl. B01D 53/38; A61L 9/00

(52) U.S. Cl. 422/4; 422/168; 422/176; 422/180

(57) ABSTRACT

An apparatus (101) for de-odorising a gas mixture, such as foul air, comprising means to provide interacting a supply of foul air (107) with a supply of oxidising liquid (106), such as ozonated water, by increasing the pressure on the foul air and increasing the surface area of the oxidising liquid.

### FIELD OF THE INVENTION

The present invention relates to the field of odour removal apparatus for foul air.

### BACKGROUND

Foul air from industrial processes, sewages, decomposition beds, etc. released into the atmosphere causes smell pollution. Odorous gases in foul air may include volatile organic compounds (VOCs), hydrogen sulphide (H₂S) and so on.

Methods of removing such odorous gases from foul air include gas adsorption, catalytic decomposition and so on.

Adsorption relies on a greater molecular affinity between the odorous gases and an adsorbent than between air and the adsorbent. An example of such an adsorbent is activated carbon. However, such adsorbents require tedious maintenance, such as frequent regeneration by applying heat to flush adsorbed molecules.

Catalytic converters are expensive. Furthermore, catalytic converters may even promote odours if large VOC molecules are partially broken down into smaller VOC molecules, instead of being fully oxidised. Thus, catalytic converters are not always reliable in cleansing foul air.

To meet the above problems, ozone has been used to oxidise odorous gases in foul air. Ozone is highly oxidising and as it is an unstable molecule, it advantageously breaks down into harmless oxygen over time. Furthermore, ozone is readily available by irradiating oxygen and can be supplied continuously and at low cost. One particular method applies ozone into the surroundings where foul air is detected. However, ozone is highly corrosive and therefore the amount useable is limited by law to not more than 0.1 ppm. Thus, this method cannot be used on a large scale. Furthermore, this method is hard to control as its performance depends on air movements.

Alternatively, ozonated water has been used. Generally, foul air is fanned across a structure supplying free-falling ozonated water to oxidise odorous gases in the foul air. However, this method relies on the extent of exposure of the foul air to the ozonated water. Allowing the foul air to interact freely with ozonated water is not sufficiently controllable to be effective in oxidising most of the odorous gases in the foul air.

Accordingly it is desirable to provide an apparatus and/or method having improved efficiency in cleansing foul air of odorous gases.

### STATEMENT OF INVENTION

In a first aspect, the invention proposes a method of de-odorising a gas mixture comprising the steps of providing an oxidising liquid to a container, providing an odorous gas mixture to the container, such that the odorous gas mixture is pressurised in the container, wherein the odorous gas mixture interacts with the oxidising liquid under pressure.

In a second aspect, the invention proposes a de-odorising apparatus for de-odorising a gas mixture comprising a container, a supply of an oxidising liquid, a gas inlet arranged to introduce an odorous gas mixture in the container, the container being configured to maintain the odorous gas mixture in the container in a pressurised state, such that the odorous gas mixture interacts with the oxidising liquid under pressure.

In a third aspect, the invention proposes a de-odorising apparatus for de-odorising a gas mixture, comprising a container, means arranged to supply an oxidising liquid to the container, means arranged to introduce an odorous gas mixture in the container, the container having means arranged to form the oxidising liquid into a liquid-film, such that the odorous gas mixture interacts with the surface of the liquid-film of oxidising liquid.

### BRIEF DESCRIPTION OF THE DRAWINGS

Embodiments of the invention will now be described, by way of example, with reference to the accompanying drawings, in which like numerals refer to like parts and in which

FIG. 1 shows an embodiment of the invention;

FIG. 1a shows the embodiment of FIG. 1 when the embodiment is not in full operation;

FIG. 1b shows a variation of the embodiment of FIG. 1;

FIG. 2 is a magnified cross-sectional view of a portion of the embodiment of FIG. 1;

FIG. 3 shows a variation to the embodiment of FIG. 1;

FIG. 3a also shows a variation to the embodiment of FIG. 1;

FIG. 4 shows a further variation of the embodiment of FIG. 1;

FIG. 5 shows another embodiment of the invention;

FIG. 5a shows yet another embodiment of the invention; and

FIG. 6 shows a further variation of the embodiment of FIG. 1.

### DETAILED DESCRIPTION

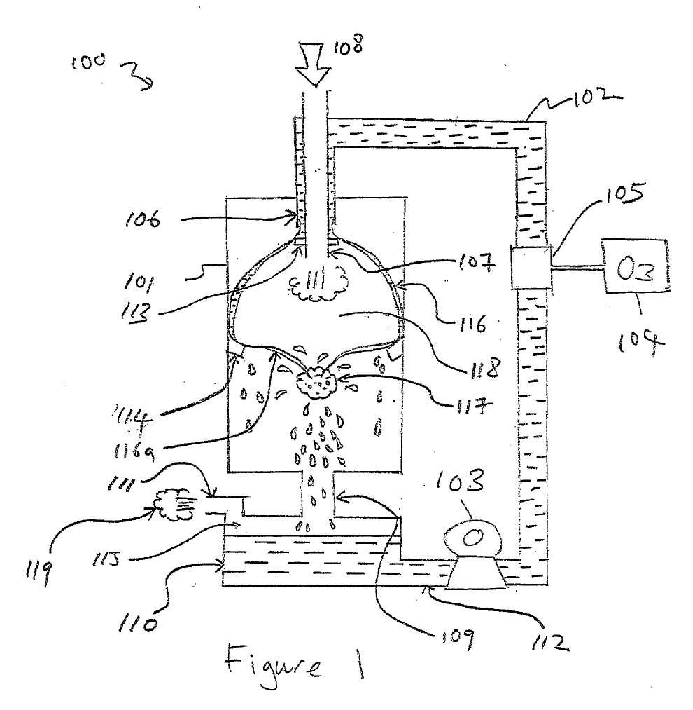

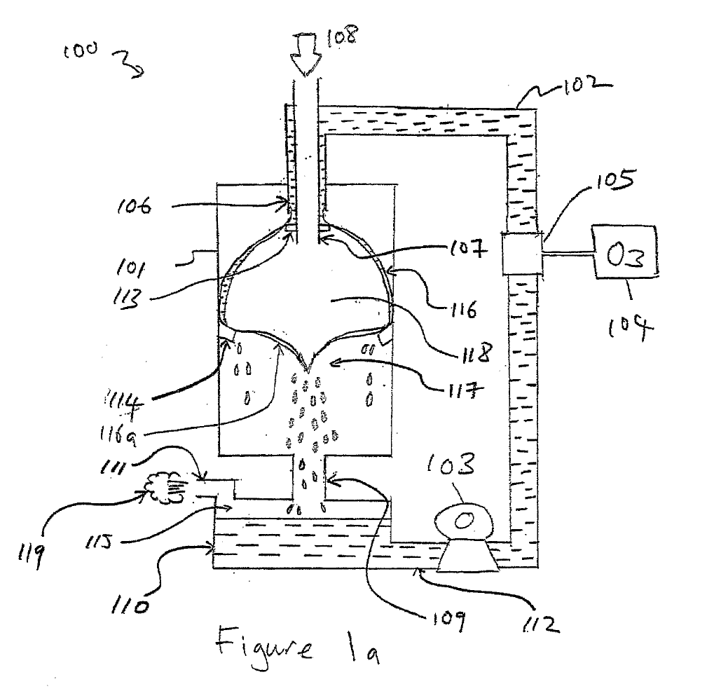

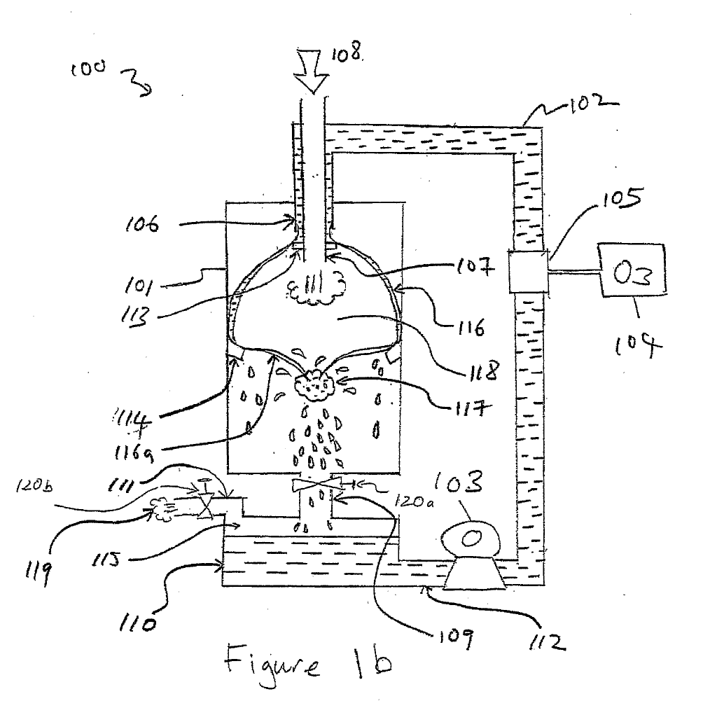

FIG. 1 illustrates an embodiment 100 of the invention, which comprises a tank 101, a set of pipings 102, a reservoir 115, a pump 103, an ozone generator 104 and a Venturi valve 105.

The tank 101 has a gas inlet 107, a water inlet 106 and a tank outlet 109.

The gas inlet 107 is connected to a fan (not shown) for drawing foul air 108 into the tank 101. Preferably, the fan is of the type used conventionally as an exhaust fan, or other types of fans capable of pressuring the tank 101 with the foul air 108. The gas inlet 107 protrudes from within the water inlet 106 to beyond the mouth of the water inlet 106 such that the water inlet 106 surrounds the gas inlet 107. A lip 113 is fashioned around the external surface of the gas inlet. The water inlet 106 is connected to the pipings 102 to draw water into the tank 101.

The tank outlet 109 is connected to a reservoir 110 beneath the tank 101. The reservoir 110 has a gas outlet 111 and a water outlet 112. The water outlet 112 is connected to the pipings 102, which is in turn connected to the water inlet 106 in the tank 101. Thus, pipings 102 provides a recycling path from the tank 101 to the reservoir 110 and back.

The reservoir 110 is filled with a pre-determined amount of water such that a headspace 115 of air is provided above the water in the reservoir 110. The gas outlet 111 of the reservoir 110 is positioned to allow air in the headspace 115 to escape through the gas outlet 111.

When the embodiment is in operation, the pump 103 draws water from the reservoir 110 into the pipings 102 and releases the water though the water inlet 106 into the tank 101. Once inside the tank, the water flows through the tank outlet 109 and re-collects in the reservoir 110 to recycle through the pipings 102.

The ozone generator 104 is positioned along the pipings 102 such that the water moving in the pipings 102 draws ozone from the ozone generator into the water by Venturi action, via the Venturi valve 105.

When the ozonated water is introduced into the tank 101, the ozonated water flows through the water inlet 106, down the external surface of the gas inlet 107 and is directed by the lip 113 to fan out radially. In other words, the ozonated water entering the tank is guided to form a thin canopy of ozonated water, i.e. a flowing water film 116, spreading and falling within the tank 101.

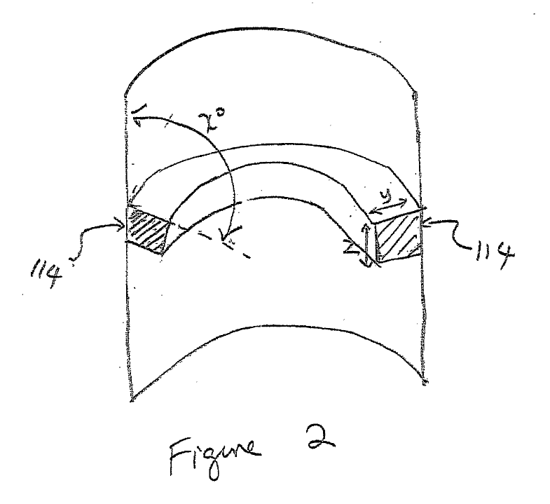

The water film 116 is directed to fall smoothly onto a deflector 114. The deflector 114 is in the form of a ledge on the internal wall of the tank 101 and is positioned mid-height in the tank 101 and extends all round the internal radial perimeter of the tank 101. FIG. 2 is a magnified sidewise cross-sectional view of the deflector 114. Furthermore, FIG. 2 shows the tank 101 having a round cross-section from the top, such that the deflector 114 lining the internal circumference of the tank 101 resembles a ring.

The deflector 114 deflects the water film 116, such that the water film 116a converges at a convergence point 117 in the centre of the tank 101. There, the water film 116a converges and then falls freely towards and through the outlet 109 of the tank 101, into the reservoir 110. Therefore, the water film 116, 116a virtually envelopes an air space 118 in the tank 101, except for the gas inlet 107 introducing the foul air 108.

To provide the deflection of the water film 116, preferably, the deflecting surface, y, and its thickness 12, depends on the applications. Furthermore, it is also preferable the deflector 114 surface is angled at 90° to the vertical surface of the tank 101 wall.

To purify a source of foul air, such as that from a sewage system, the foul air 108 is drawn into the tank 101 by the exhaust fan (not shown) through the gas inlet 107 and is released inside the water film-enveloped air space 118. The foul air is supplied continuously such that the air space 118 enveloped by the water film becomes pressurised. The pressure causes the foul air 108 molecules to become more concentrated in the air space, which therefore interact efficiently with the ozonated water to oxidise the odorous gases in the foul air. In other words, the pressurisation of the foul air 108 improves the likelihood of molecular interaction and, consequently, oxidation of odorous gases in the foul air.

Furthermore, some of the odorous gases are pressured to dissolve into the ozonated water, which further improves their likelihood of being oxidised.

Spreading the ozonated water to form a water film 116 provides a greater water surface area than if the ozonated water is allowed to free-fall as water droplets. Therefore, the ozonated water is exposed as fully as possible to the pressurised foul air 108 in the air space 118. This further improves interaction between the ozonated water and the foul air, promoting efficient oxidation of the odorous gases in the foul air.

Furthermore, as ozone is not too highly soluble in water, a portion of the ozone in the ozonated water escapes as ozone gas into the air space 118 (any undissolved ozone bubbles carried from the ozone generator 104 is also released into the air space 118). Thus, the foul air 108 in the air space 118 is also continuously mixed with ozone gas. This further ensures oxidation of odorous gases in the foul air 108.

The flowing water film 116 has a surface tension that resists being broken easily by the foul air 108. Thus, the pressure in the tank has to build up to a certain level before the foul air 108 can be pressurised to break though the water film at the convergence point 117. It has been detected experimentally that there is a pressure loss between the supply of foul air 118 and the release of cleansed air from the reservoir, which accounts for the energy used to break through the convergence point 117. Thus, at the convergence point 117, ozonated water, ozone gas and air mix chaotically, which further oxidises any remaining odorous gases in the foul air 108.

FIG. 1a illustrates the convergence point 117 if it is not broken by the pressurised flow of foul air. However, during operation, it is not always possible to see an unbroken convergence point 117, as the water film 116 at the convergence point 117 is broken chaotically and frequently by the continuously supply of pressurised foul air.

From the convergence point, the falling ozonated water and cleansed air 119 moves downwardly and passes through the tank outlet 109 to accumulate in the reservoir 110. In the reservoir 110, the cleansed air 119 and the water are allowed to settle and separate. Residual ozone in the headspace and the water oxidises residual odorous compounds in the reservoir 110, if there is any.

From the reservoir, the cleansed air 119 leaves the reservoir 110 through the gas outlet 111, propelled by the pressure exerted by the continuous introduction of foul air 108 into the tank 101. The water in the reservoir 110 is sucked by the pump 103 into recycling through the pipings 102 and mixing with the foul air 108 in the tank 101.

Preferably, the outlet 109 is dimensioned to maintain a predetermined amount of pressure in the tank 101, such that the environment in the tank is also slightly pressurised, although not as much as in the air space enveloped by the water film. This possible because the outlet 109 provides an output restriction which eventually creates a stable tank pressure at equilibrium.

FIG. 1b shows in combination two possible variations of the embodiment of FIG. 1. The embodiment may be fitted with a valve 120a for adjusting the tank outlet 109 and to control the pressure in the tank 101. Alternatively, the valve 120b may be placed at the outlet of the reservoir, which will allow the pressure in the headspace 115 to be controlled as well.

Therefore, the embodiment provides a method of de-odorising a gas mixture 108 comprising the steps of providing an oxidising liquid into a container 101, providing an odorous gas mixture 108 in the container 101, such that the odorous gas mixture 108 is pressurised in the container 101; wherein the odorous gas mixture 108 interacts with the oxidising liquid under pressure.

Furthermore, the embodiment provides a method of de-odorising a gas mixture 108 comprising the steps of providing an oxidising liquid, forming the oxidising liquid into a liquid-film 116, providing an odorous gas mixture 108, such that the odorous gas mixture 108 interacts with the surface of the liquid-film 116 of the oxidising liquid.

Furthermore, the embodiment provides a de-odorising apparatus 100 for de-odorising a gas mixture 108 comprising a container 101, a water introduction means 102, 106 for introducing water into the container 101, an ozone generator 104 arranged to introduce ozone into the water to form ozonated water, the container 101 further having a gas inlet 107 arranged to introduce an odorous gas mixture 108 in the container 101, the container 101 configured to maintain the odorous gas mixture 108 in the container 101 pressurised, such that the odorous gas mixture 108 interacts with the ozonated water under pressure.

Furthermore, the embodiment is a de-odorising apparatus 100 for de-odorising a gas mixture 108 comprising a container 101, a means 104 for providing an oxidising liquid, the container 101 having a liquid inlet 106 arranged to introduce the oxidising liquid into the container 101, the container 101 further having a gas inlet 107 arranged to introduce an odorous gas mixture 108 in the container 101, the container 101 having a means 113 for forming the oxidising liquid into a liquid-film 116, such that the odorous gas mixture 108 interacts with the surface of the liquid-film of oxidising liquid 116.

Therefore, the embodiment provides a mixing environment that is highly pressurised, a large interaction area between foul air and ozonated water and also chaotic mixing. This allows ozone to oxidise odorous gases in the foul air more effectively than merely sprinkling ozonated water in a cross-flowing gust of foul air.

### Exemplary Experiment Data

As shown in Table 1, it has been found experimentally that foul air 108 containing 440 ppb of hydrogen sulphide (H₂S) drawn into a prototype of an embodiment of the invention at a flowrate of 1.8 m³/minute has a cleansed air 119 output of 0 ppb hydrogen sulphide, i.e. 100% removal of hydrogen sulphide.

In a second experiment, it has been found experimentally that foul air 108 containing 1800 ppb of hydrogen sulphide (H₂S) drawn into a prototype of an embodiment of the invention at a flowrate of 1.8 m³/minute removes the hydrogen sulphide by 87%.

TABLE I

| | | Reduction of H₂S content | Introduction rate |
|---|---|---|---|
| | Initial | after mixing with ozonated | of foul air 108 |
| | H₂S | water in a prototype | into the prototype |
| | content | of the embodiment. | (m³/min). |
| Experiment | 440 ppb | 100% | 1.8 |
| 1 | | | |
| Experiment | 1800 ppb | 87% | 1.8 |
| 2 | | | |

### Conditions of the Experiment:

Tank size: 600 L
Water content in tank maintained at 300 L
Flowrate of the recycled ozonated water: 50 L/min
Ozone introduction flowrate: 5 gm/hr at 6% by weight, 3-6 L/min

### Ambient Temperature: 33° C.

Relative humidity: 80%

The experiment results may be further improved depending on the flowrate of the ozonated water, the quantity of the ozone carried by the ozonated water, the flowrate of the foul air 108, the pressure of the foul air 108 in the tank 101. Furthermore, the experiment results may be further improved depending the hydrodynamics of the water film 116, which is determined, inter alia, by the height of the tank 101, the diameter of the tank 101, the flowrate and pressure of the water input from the water inlet 106, the surface energy of the material used to construct the tank 101, the positioning and dimensions of the deflector 114, the positioning and dimension of the lip 113 and so on. These factors are variable according to the scale of the implementation of the embodiments and according to specific performance requirements. Therefore, preferably, the tank 101 is made of transparent glass or plastics material, which is resistant to the corrosive action of ozone and so that the process may be viewed from the outside of the tank 101. This provides easy adjustment, maintenance and repair planning based on visual observation.

In one variation of the embodiment, the water film 116 may be directed to flow down the internal surface of the tank 101 instead of free falling as a water film, before being re-directed by the deflector 114. Furthermore, instead of the lip 113, other means of fanning the ozonated water, such a suitable arrangement of water jets, may be used to form the film of ozonated water.

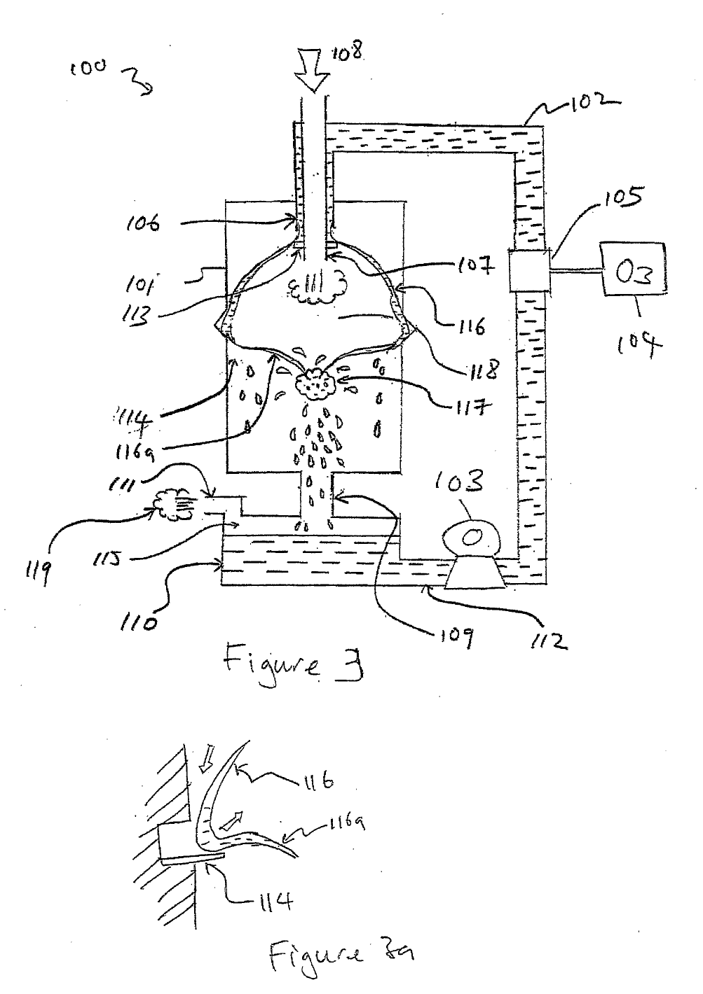

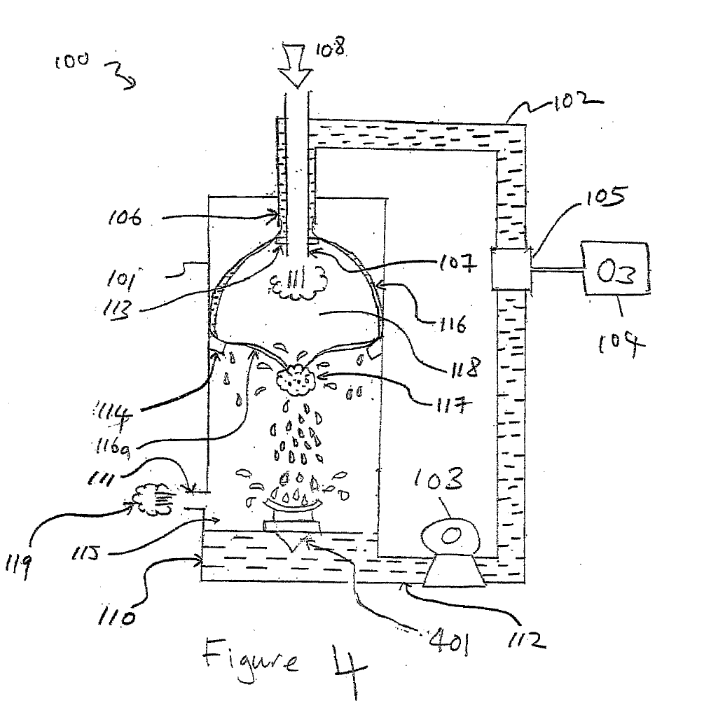

Furthermore, the angle, shape and other dimensions of the deflector 114 may be varied, depending on the desired deflection characteristics of the water film 116. FIG. 3 shows a variation of the embodiment, having a kink or a groove in the internal surface of the tank 101 providing the deflector 114, instead of a protruding ledge. FIG. 3a shows groove in the tank wall having an attached protruding ledge that deflects the water film 116 according to the arrows shown in FIG. 3a towards the convergence point 117 (no shown in FIG. 3a).

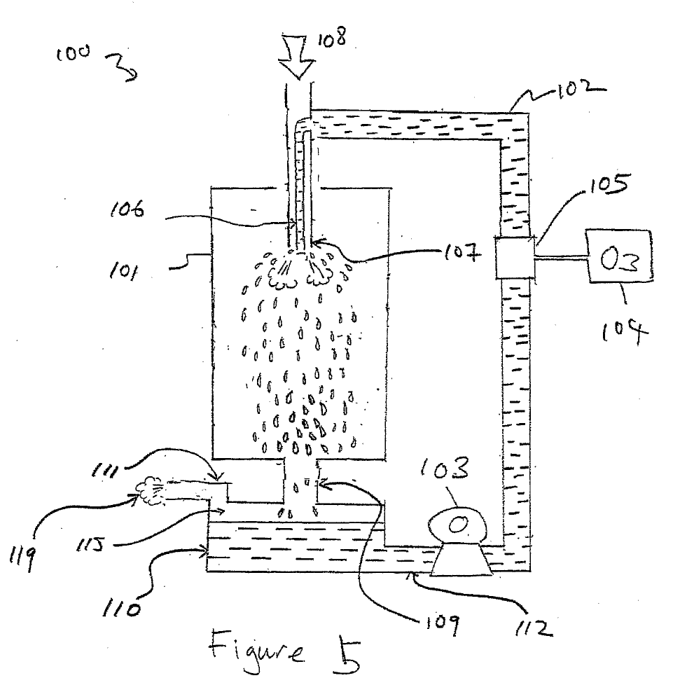

FIG. 4 shows a further variation of the embodiment, in which a float 401 is provided in the reservoir 110 which floats on the surface of accumulated ozonated water in the tank and positioned such that the down-pouring water from the convergence point 117, carrying some of the air from the air space, splashes on the float 401. The splashing creates further chaotic interaction between the air, ozone and ozonated water in the reservoir 110 and improves the efficiency of oxidising odorous components. In this variation of the embodiment, the tank outlet 109 is removed.

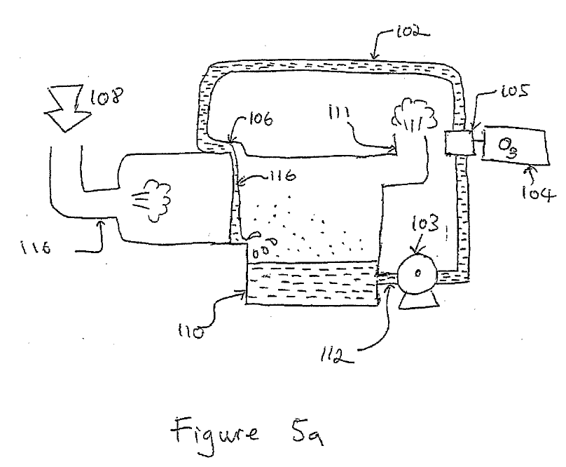

FIG. 5 shows another embodiment of the invention, which is structurally simpler and useable when the foul air-ozonated water interaction does not have to be as intense in the above-described embodiments to provide good oxidation efficiency. For example, when the amount of odorous gases is very high and the embodiment is used as a preliminary stage de-odorising apparatus, before a series of further deodorising treatments.

In the embodiment of FIG. 5, the ozonated water is introduced to fall freely, for example, to form a light spray or a heavy downpour, instead of forming a film. Thus, it is possible but not necessary to have a deflector for deflecting the ozonated water. Furthermore, the water inlet may be positioned inside the gas inlet, as opposed to the arrangement in the earlier embodiments, so that the ozonated water is well dispersed among the foul air 108. The foul air is, however, still introduced into the tank 101 such that the foul air 108 in the tank 101 is pressurised.

The effectiveness of this embodiment may depend on the pressure of the foul air, flowrate of the ozonated water and the foul air into the tank; the higher the flowrate of the ozonated and the lower the flowrate of the foul air, the more ozonated water the foul air is exposed to.

A variation to the embodiment of FIG. 5 is that the foul air is introduced sidewise of the tank and to flow laterally across the spray of ozonated water.

Yet a further variation to the embodiment of FIG. 5 is that the falling ozonated water and foul air is directed to impact on a surface (not illustrated), such as a float as shown in FIG. 4.

In another structurally simple application, illustrated in FIG. 5a, foul air 108 may be introduced to pass though a planar screen of ozonated water, i.e. from one side to the other side of a water film 116 that flows down in the path of the foul air, instead of being entrapped in a surrounding canopy of water film. The movement of the foul air 108 being initially resisted by the water film 116 is eventually forced to break through the water film 116 and in the processes interacts with the ozone in the water under increased pressure.

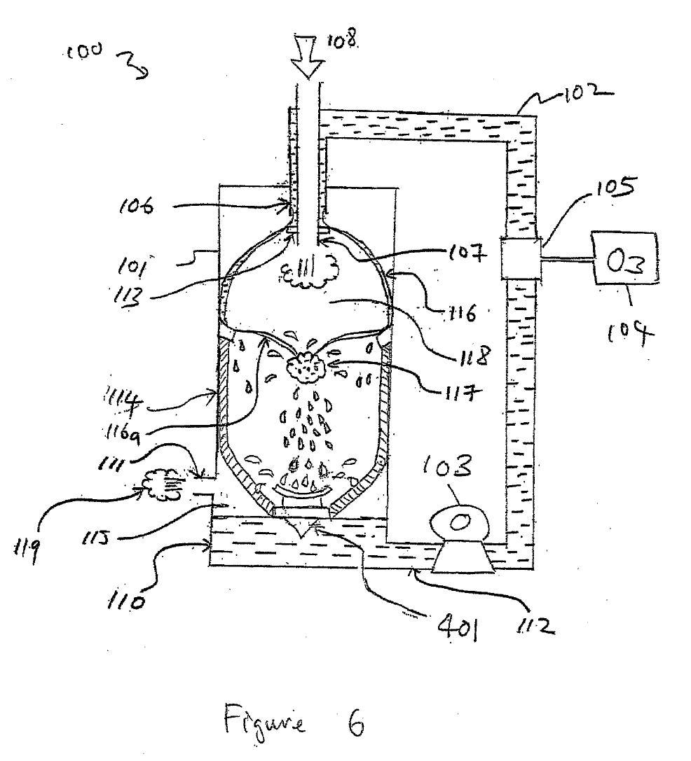

FIG. 6 shows yet a further variation of the embodiments of FIG. 1 and FIG. 4, in which the deflector 114 is supported by a supporting float 104 on the surface of the accumulated ozonated water in the reservoir 110. The height of the deflector 114 is therefore adjustable by controlling the level of accumulated water in the reservoir 110. Furthermore, it is not necessary but preferable that the float 104 provides a surface against which down pouring ozonated water splashes, to further promote interaction. One advantage provided by the embodiments is that the embodiments may be implemented without needing elaborate aerodynamic or airflow designs, as in the case of activated carbon beds. Accordingly, the embodiments are easier and quicker to design and install.

A further advantage of the embodiments is the removal of soluble gases by dissolution into the water, whether the gases are oxidisable by the ozone or not. Thus, airborne corrosive substances, such as salt, may be trapped in the water and thus removed from the cleansed air 119.

Furthermore, it is also possible to use the embodiments to mix particulate-laden air and water to retain the particulates in the water, while releasing the water-filtered air. In other words, it is also possible to oxidise airborne but non-gaseous contaminants.

Furthermore, ozone kills bacteria and microorganisms. Thus, the embodiments further provide the advantageous possibility of some sterilisation of the foul air 108.

A further advantage of the embodiments is the possibility of a continuous process, in contrast to the batch process of the activated carbon beds of the prior art. Furthermore, the embodiment is not limited to being used as a single unit; it is possible to form several cascading installations of the embodiment to oxidise the foul air 108 in consecutive stages.

Furthermore, the embodiments are useable to cleanse gases other than foul air. For example, any gas that is stable in the presence of ozone can be cleansed of oxidisable contaminants. Furthermore, other oxidising substances can be used in place of ozonated water, such as bleach.

Thus, the described embodiments provide the possibility of continuously supplying a clean source of clean air, cleansed of odorous gases, volatile organic compounds, particulates, micro-organisms and so on.

1. A method of de-odorising a gas mixture comprising the steps of:
providing an oxidising liquid to a container;
providing an odorous gas mixture to the container, such that the odorous gas mixture is pressurised in the container; wherein
the odorous gas mixture interacts with the oxidising liquid under pressure.

2. A method of de-odorising a gas mixture as claimed in claim 1 wherein the oxidising liquid is supplied continuously; and the odorous gas mixture is supplied continuously.

3. A method of de-odorising a gas mixture as claimed in claim 1, further comprising the step of forming the oxidising liquid into a liquid film; and pressurising the odorous gas mixture on one side of the liquid film.

4. A method of de-odorising a gas mixture as claimed in claim 3, further comprising the step of deflecting the liquid film to converge at a point of convergence; wherein the odorous gas mixture crosses the liquid film at the point of convergence.

5. A method of de-odorising a gas mixture as claimed in claim 4, further comprising the step of directing the oxidising liquid and the odorous gas mixture from the point of convergence to impact onto a surface.

6. A method of de-odorising a gas mixture comprising the steps of:
providing an oxidising liquid;
forming the oxidising liquid into a liquid-film of oxidising liquid;
providing an odorous gas mixture; such that the odorous gas mixture interacts with the surface of the liquid-film of oxidising liquid.

7. A method of de-odorising a gas mixture as claimed in claim 6, further comprising the step of directing the liquid-film such that the liquid-film forms a gas entrapment space for receiving the odorous gas mixture.

8. A de-odorising apparatus for de-odorising a gas mixture comprising:
a container,
a supply of an oxidising liquid;
a gas inlet arranged to introduce an odorous gas mixture in the container;
the container being configured to maintain the odorous gas mixture in the container in a pressurised state; such that the odorous gas mixture interacts with the oxidising liquid under pressure.

9. A de-odorising apparatus for de-odorising a gas mixture as claimed in claim 8 wherein the supply of oxidising liquid is continuous; and the supply of the odorous gas mixture is continuous.

10. A de-odorising apparatus for de-odorising a gas mixture as claimed in claim 8 further comprising means for forming the oxidising liquid into a liquid-film of oxidising liquid.

11. A de-odorising apparatus for de-odorising a gas mixture as claimed in claim 10 wherein the liquid-film of oxidising liquid defines a gas entrapment space for receiving the pressurised odorous gas mixture.

12. A de-odorising apparatus for de-odorising a gas mixture as claimed in claim 11 wherein the odorous gas mixture is pressurised such that the odorous gas mixture crosses the liquid-film of oxidising liquid.

13. A de-odorising apparatus for de-odorising a gas mixture as claimed in claim 12 wherein the container further comprising a deflector for deflecting the liquid-film of oxidising liquid such that the liquid-film of oxidising liquid converges at a point of convergence, and the odorous gas mixture is pressurised such that the odorous gas mixture crosses the liquid-film of ozonated water at through the point of convergence.

14. A de-odorising apparatus for de-odorising a gas mixture as claimed in claim 8 wherein the container contains a float capable of floating on oxidising liquid accumulated in the container; the float providing a surface on which the odorous gas mixture and oxidising liquid is directed to impact.

15. A de-odorising apparatus for de-odorising a gas mixture as claimed in claim 13 wherein the deflector is supported by a float capable of floating on oxidising liquid accumulated in the container.

16. A de-odorising apparatus for de-odorising a gas mixture comprising
a container,
means arranged to supply an oxidising liquid to the container;
means arranged to introduce an odorous gas mixture in the container;
the container having means arranged to form the oxidising liquid into a liquid-film;
such that the odorous gas mixture interacts with the surface of the liquid-film of oxidising liquid.

17. A de-odorising apparatus for de-odorising a gas mixture as claimed in claim 16 wherein the liquid-film defines a gas entrapment space for containing the odorous gas mixture.
